(19) United States
(12) Patent Application Publication
(10) Pub. No.: US 2019/0142447 A1
(43) Pub. Date: May 16, 2019

(54) TRANSAPICAL DELIVERY MITRAL VALVE PROCEDURE DEVICES

(71) Applicants: North Carolina State University, Raleigh, NC (US); The University of North Carolina at Chapel Hill, Chapel Hill, NC (US)

(72) Inventors: Ke Cheng, Raleigh, NC (US); Thomas George Caranasos, Chapel Hill, NC (US); Adam Vandergriff, Raleigh, NC (US)

(21) Appl. No.: 16/098,671

(22) PCT Filed: May 5, 2017
(86) PCT No.: PCT/US2017/031241

§ 371 Date: Nov. 2, 2018

Related U.S. Application Data
(60) Provisional application No. 62/332,854, filed on May 6, 2016.

(51) Int. Cl. A61B 17/28; A61F 2/24; A61B 17/295; A61B 17/068

(52) U.S. Cl. CPC A61B 17/2841; A61F 2/2466; A61B 2017/00292; A61B 17/068; A61B 17/2833; A61B 17/295

(57) ABSTRACT

Various embodiments of a device for mitral valve repair are described. In one example, the device can be used to place a pair of magnets between the flaps of the mitral valve. The magnets hold the flaps together near its center, with a result similar to that achieved through the Alfieri stitch. To place the magnets, the device is inserted through the apex of the heart in a transapical procedure and extended up into the left ventricle toward the mitral valve. When in position with the mitral valve, a pair of grasper fingers of the device are used to place the pair of magnets over the flaps, and the magnets are released. Once released, magnetic attraction between the magnets holds the flaps together. In other embodiments, the device can be used to place staples or sutures or other bonding elements over the flaps of the mitral valve.

### CROSS-REFERENCE TO RELATED APPLICATIONS

This application claims the benefit of U.S. Provisional Application No. 62/332,854, filed May 6, 2016, the entire contents of which is hereby incorporated herein by reference.

### BACKGROUND

Mitral insufficiency, regurgitation, or incompetence is a common form of valvular heart disease found in humans, dogs, and other mammals in which the heart mitral valve does close sufficiently when pumping. The disorder leads to blood backwards from the left ventricle, through the mitral valve, and into the left atrium. This backwards blood leakage often leads to increased blood pressure in the left atrium and pulmonary veins. Other symptoms include fatigue, labored breathing, coughing, and a rapid fluttering heartbeat among others. If regurgitation is severe, fluid can build up in the lungs, and can lead to congestive heart failure.

### SUMMARY

Various embodiments of a device and process for mitral valve repair are described. In one embodiment, a device for a transapical procedure to mitigate mitral regurgitation includes a handle assembly, a cannula assembly, and a valve procedure tool. The valve procedure tool can be disposed at a distal end of the cannula assembly to position and place a bonding element to flaps of a mitral valve during the transapical procedure.

In other aspects, the handle assembly can include a handle grip, a handle trigger, and pivot hardware that secures the handle trigger in position as a lever with the handle grip. Further, the cannula assembly can include a cannula cover tube and a cannula rotator knob to rotate the cannula cover tube and the valve procedure tool. Additionally, the valve procedure tool can include at least one grasper finger to place the bonding element to the flaps of the mitral valve during the transapical procedure.

In one case, the bonding element can include a plurality of magnets, and the valve procedure tool can include a plurality of grasper fingers to place the plurality of magnets over the flaps of the mitral valve during the transapical procedure. The plurality of magnets can be enclosed within the plurality of grasper fingers. In that case, the device can include a sliding door in each of the plurality of grasper fingers to release the plurality of magnets from the device over the flaps of the mitral valve during the transapical procedure.

In other examples, the bonding element can include at least one of a magnet, a staple, or suture filaments, and the valve procedure tool can include a plurality of grasper fingers to place the bonding element over the flaps of the mitral valve during the transapical procedure.

In another embodiment, a transapical procedure to mitigate mitral regurgitation is described. The procedure can include inserting a cannula assembly of a device into an apex opening in a heart, extending the cannula assembly of the device through the heart to approach a mitral valve of the heart, and positioning a valve procedure tool about flaps of a mitral valve.

Positioning the valve procedure tool can include rotating the valve procedure tool about the flaps of the mitral valve. After positioning the valve procedure tool, the procedure can also include pressing at least a portion of the flaps of the mitral valve together. Pressing at least the portion of the flaps of the mitral valve together can include bonding at least a portion of the flaps of the mitral valve together by applying a bonding element to the flaps of the mitral valve using the valve procedure tool.

The bonding element can include at least one of a magnet, a staple, or suture filaments, and the valve procedure tool can include a plurality of grasper fingers to place the bonding element over the flaps of the mitral valve during the transapical procedure. In another example, the bonding element can include a plurality of magnets, and the valve procedure tool can include a plurality of grasper fingers to place the plurality of magnets over the flaps of the mitral valve during the transapical procedure.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 illustrates an example device for a mitral valve procedure according to various embodiments described herein.

FIG. 2 illustrates an example illustration of a heart for a mitral valve procedure according to various embodiments described herein.

FIGS. 3A-3C illustrate example components of a cannula assembly of the device shown in FIG. 1 according to various embodiments described herein.

FIGS. 4A and 4B illustrate example components of a grasper door release assembly of the device shown in FIG. 1 according to various embodiments described herein.

FIGS. 5A and 5B illustrate example components of a grasper assembly of the device shown in FIG. 1 according to various embodiments described herein.

FIG. 6 illustrates an example process for a transapical procedure according to various embodiments described herein.

The drawings illustrate only example embodiments and are therefore not to be considered limiting of the scope of the embodiments described herein, as other embodiments are within the scope of this disclosure. The elements and features shown in the drawings are not necessarily drawn to scale, emphasis instead being placed upon clearly illustrating the principles of the embodiments. Additionally, certain dimensions or positionings may be exaggerated to help visually convey certain principles. In the drawings, similar reference numerals between figures designate like or corresponding, but not necessarily the same, elements.

### DETAILED DESCRIPTION

As noted above, mitral insufficiency, mitral regurgitation, or incompetence (collectively “MR”) is a common form of valvular heart disease found in humans and dogs. The disorder leads to blood that flows backwards from the left ventricle, through the mitral valve, and into the left atrium. This backwards blood leakage often leads to increased blood pressure in the left atrium and pulmonary veins. MR can be diagnosed using chest X-rays, echocardiography, electrocardiography, and/or other diagnostic techniques.

Methods of indirect treatment, such as angiotensin-converting-enzyme (ACE) inhibitors to reduce blood pressure, diuretics to reduce fluid retention, and digoxin to improve heart function and hormone release, can offer symptom management, but not a solution to the problem of MR. Methods of direct treatment include the Alfieri stitch, the MitraClip® device, and other devices and techniques. Direct methods of treatment offer a better long-term solution to MR, beyond symptom management.

In the context outlined above, the embodiments described herein are directed to a transapical delivery device for the treatment of MR and mitral valve leakage. The device provides an alternative to conventional MR mitigation techniques and devices, including the Alfieri stitch, the MitraClip® device, and other devices and techniques. The device is more cost effective, less evasive, and easier to use than other devices available on the market. For example, the MitraClip® device must be delivered through the leg (i.e., transfemoral delivery) in a lengthy and costly procedure. Thus, the MitraClip® device is prohibitively expensive for use by veterinary surgeons to treat companion animals. In contrast to the MitraClip® device and its method of use, the device described herein can be deployed transapically in a less costly procedure.

In one embodiment, the device described herein can be used to place a pair of magnets between the two cusps, leaflets, or flaps of the mitral valve. When placed correctly, the two magnets hold the flaps of the mitral valve together near its center, with a result similar to that achieved through the more evasive Alfieri stitch. To place the magnets, the device is inserted through the apex of the heart in a transapical procedure and extended up into the left ventricle toward the mitral valve. When the distal end of the device is in position with the mitral valve, a pair of grasper fingers of the device are used to place the pair of magnets over the flaps of the mitral valve. When the magnets have been suitably positioned (and/or repositioned) over the flaps, they are released by the device, and the magnetic attraction between the magnets presses and holds the flaps together. In other embodiments of the device, the grasper fingers can be used to place other bonding or connecting elements, such as one or more staples, sutures, or other means (rather than or in addition to magnets) over the flaps of the mitral valve.

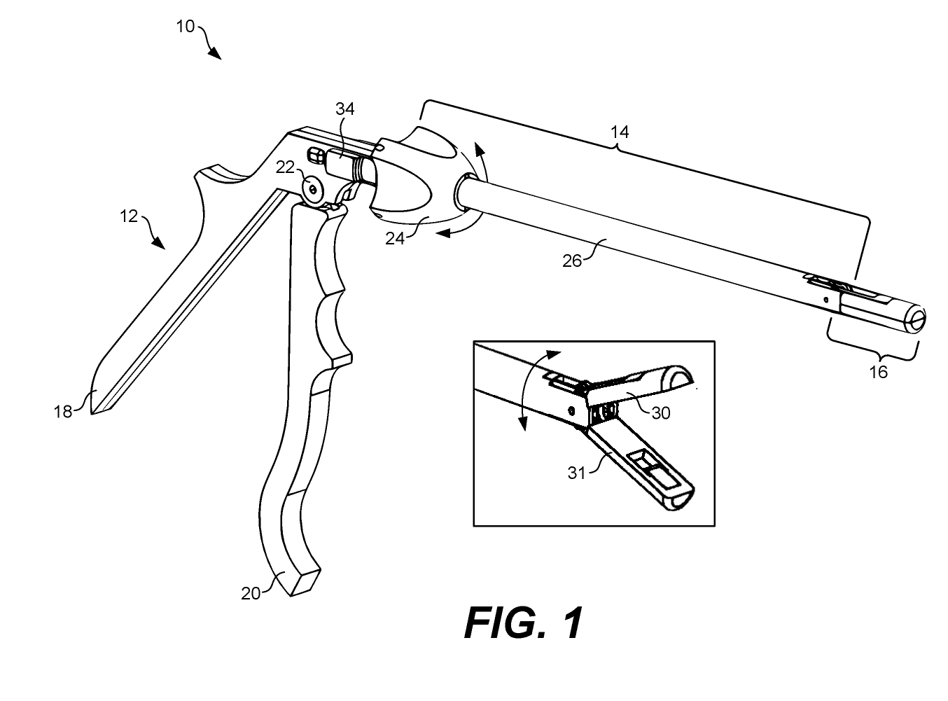

Turning to the drawings, FIG. 1 illustrates an example device 10 for a mitral valve procedure according to various embodiments described herein. The device 10 includes a handle assembly 12, a cannula assembly 14, and a valve procedure tool 16. The handle assembly 12 includes a handle grip 18, a handle trigger 20, and pivot hardware 22 that secures the handle trigger 20 in position as a lever with the handle grip 18. The cannula assembly 14 includes a cannula rotator knob 24 and a cannula cover tube 26. As shown in the inset in FIG. 1, the valve procedure tool 16 includes a first grasper finger 30 and a second grasper finger 31 which can be opened in response to pulling the handle trigger 20. Using the cannula rotator knob 24, the cannula 14 assembly, including the grasper fingers 30 and 31, can be rotated. The device 10 also includes a release slide 34 that can be used to release magnets held in the first and second grasper fingers 30 and 31.

The device 10 can be formed from any suitable type(s) of materials, including metal(s), metal alloy(s), plastic(s), rubber(s), ceramic(s), glass, other materials, and combinations thereof. In some cases, one or more parts of the device 10 can be formed from flexible material(s) while other parts are formed from more rigid materials. For example, the cannula assembly 14 (or at least a length of the cannula assembly 14) can be formed from flexible plastic(s), rubber(s), or other materials while the handle assembly 12 is formed from more rigid materials.

Because the device 10 can be configured to deliver magnets, staples, sutures, or other bonding or connecting structures means to the flaps of a mitral valve as described herein, the device 10 can also include those types of materials. When the device 10 is configured to deliver magnets, it may be formed from non-magnetic materials such as aluminum, plastic, or rubber, for example, so that the magnets can be easily released from the device 10 without magnetic interference.

In one embodiment, the device 10 is formed as a disposable, one-time-use device. In that case, the device 10 may be thrown away after being used in a medical procedure. Alternatively, the device 10 can be used in a number of different procedures and cleaned and sterilized between procedures. In either case, the device 10 can be formed from metal(s), metal alloy(s), plastic(s), rubber(s), ceramic(s), glass, other materials, and combinations thereof. The device 10 can also be formed to any suitable size based on various factors, including the type and/or size of the heart being operated upon, among other anatomical features and/or surgical factors.

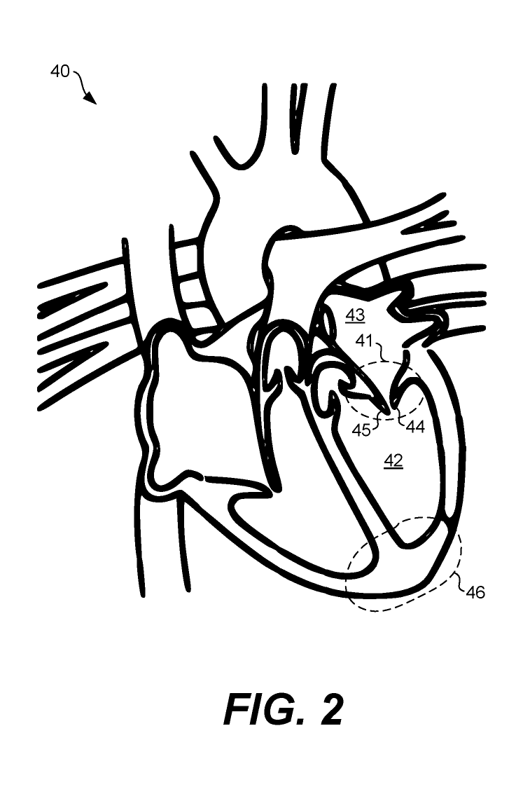

Before turning to a more detailed description of (and variations on) the device 10, an example mitral valve procedure is described using the device 10 with reference to FIG. 2. FIG. 2 illustrates an example illustration of a heart 40 including a mitral valve 41. The heart 40 is provided as a representative illustration to provide context to the description of the embodiments. The embodiments are not limited to use with any particular type and/or size of heart or even type of heart valve, however, and FIG. 2 is only an example.

As noted above, MR is a form of valvular heart disease in which the mitral valve 41 does not close sufficiently when pumping. The disorder leads to blood that flows backwards from the left ventricle 42, through the mitral valve 41, and into the left atrium 43. This backwards blood leakage often leads to increased blood pressure in the left atrium 43 and pulmonary veins. Other symptoms include fatigue, labored breathing, coughing, and a rapid fluttering heartbeat, among other symptoms.

An example mitral valve procedure using the device 10 includes placing a pair of magnets between the two cusps, leaflets, or flaps 44 and 45 of the mitral valve 41 in the heart 40. To begin, an opening is cut through the apex 46 of the heart 40, and the cannula assembly 14 of the device 10 is inserted through the opening in a transapical procedure. The cannula 14 is extended up into the left ventricle 42 and toward the mitral valve 41. The placement of the cannula 14 within the left ventricle 42 can be confirmed during the procedure using echocardiography, electrocardiography, or another suitable diagnostic technique(s) (or combinations thereof). In some cases, a camera can also be used to identify the apex 46 or other features of the heart 40 during the procedure.

When the end of the valve procedure tool 16 is in positioned close to the flaps 44 and 45 of the mitral valve 41, the grasper fingers 30 and 31 can be opened around the flaps 44 and 45 by pulling on the handle trigger 20. At the same time, the relative positions of the device 10, the grasper fingers 30 and 31, and the flaps 44 and 45 can be confirmed using echocardiography, electrocardiography, or any other suitable diagnostic technique(s). Additionally, the cannula 14 assembly, including the grasper fingers 30 and 31, can be rotated into position around the flaps 44 and 45 using the cannula rotator knob 24.

After the grasper fingers 30 and 31 have been positioned around the flaps 44 and 45, the handle trigger 20 can be released to close the grasper fingers 30 and 31 over the flaps 44 and 45 (e.g., over central portions of the flaps 44 and 45). In some cases, with the grasper fingers 30 and 31 closed over the flaps 44 and 45, the performance of the mitral valve 41 can be evaluated using echocardiography. If needed, the grasper fingers 30 and 31 can be repositioned over the flaps 44 and 45 again to achieve better results. For example, the grasper fingers 30 and 31 may be repositioned to hold the flaps 44 and 45 together near the center of the flaps 44 and 45.

When the grasper fingers 30 and 31 are suitably positioned and closed over the flaps 44 and 45, the magnets in the grasper fingers 30 and 31 can be released using the release slide 34. As described in further detail below, when the release slide 34 is pulled back, doors in the grasper fingers 30 and 31 are also pulled or slid back, releasing the magnets out from within the grasper fingers 30 and 31. The magnets are magnetically attracted to each other and hold (e.g., press) the flaps 44 and 45 together in a manner similar to that achieved by the Alfieri stitch. In other embodiments of the device, the grasper fingers 30 and 31 can be used to place one or more staples or sutures (rather than or in addition to magnets) over the flaps of the mitral valve.

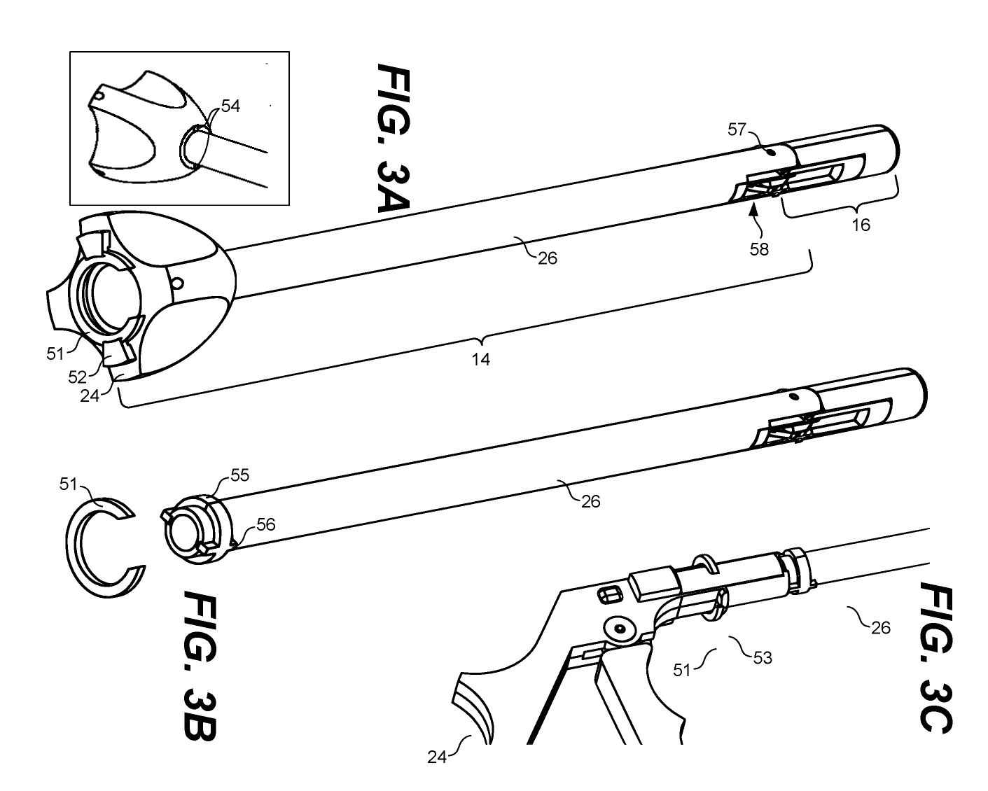

In the following paragraphs, the individual components and operation of the device 10 are described in greater detail with reference to FIGS. 3A-3B, 4A, 4B, 5A, and 5B. Starting with FIGS. 3A-3C, example components of the cannula assembly 14 of the device 10 are shown. The handle assembly 12 is omitted from view in FIG. 3A, the handle assembly 12 and the cannula rotator knob 24 are omitted from view in FIG. 3B, and the cannula rotator knob 24 is omitted from view to focus on other components in FIG. 3C. Referring to FIGS. 3A and 3B, the cannula assembly 14 includes the cannula rotator knob 24, the cannula cover tube 26, and a cannular locking washer 51.

Referring to FIG. 3A, the cannula rotator knob 24 can be conoid in shape, including fingertip depressions for rotating the cannula assembly 14, although it can be formed in other suitable shapes. The cannula rotator knob 24 includes locking clips 52 to clip over the locking washer 51. When the device 10 is assembled, the locking washer 51 abuts against the lip 53 of the handle grip 18 as shown in FIG. 3C. Because the locking clips 52 of the cannula rotator knob 24 clip over the locking washer 51, the locking washer 51 holds the cannula rotator knob 24 in place with respect to the handle grip 18 based on a mechanical interference between the locking washer 51 and the lip 53 of the handle grip 18.

Referring to the inset in FIG. 3A, the cannula rotator knob 24 includes rotational transfer slits 54 at its tapered end. As shown in FIG. 3B, a cannula rotator clip 55, including rotational transfer notches 56, transfers rotational forces from the cannula rotator knob 24 to the cannula cover tube 26. The rotator clip 55, which may be formed as two symmetrical, semicircular halves, is securely seated into a recess formed into the cannula cover tube 26 at one end. When the device 10 is assembled, the cannula rotator knob 24 fits over the rotator clip 55, with the rotational transfer notches 56 of the rotator clip 55 fitting securely into the rotational transfer slits 54 of the cannula rotator knob 24. In that configuration, when the cannula rotator knob 24 is twisted, rotational force is transferred from the cannula rotator knob 24 to the cannula cover tube 26 through the cannula rotator clip 55 and rotational transfer notches 56. Based on the structure of the device 10, rotation of the cannula rotator knob 24 leads to rotation of the cannula assembly 14 and the valve procedure tool 16 as described herein.

As shown in FIGS. 3A and 3B, the cannula cover tube 26 also includes a grasper pivot shaft hole 57 and a grasper expansion channel 58 at its distal end. A pivot shaft is inserted into the pivot shaft hole 57 and permits the grasper fingers 30 and 31 to pivot into an opened position (e.g., as shown in the inset in FIG. 1). The grasper expansion channel 58 permits clearance for the grasper fingers 30 and 31 to open.

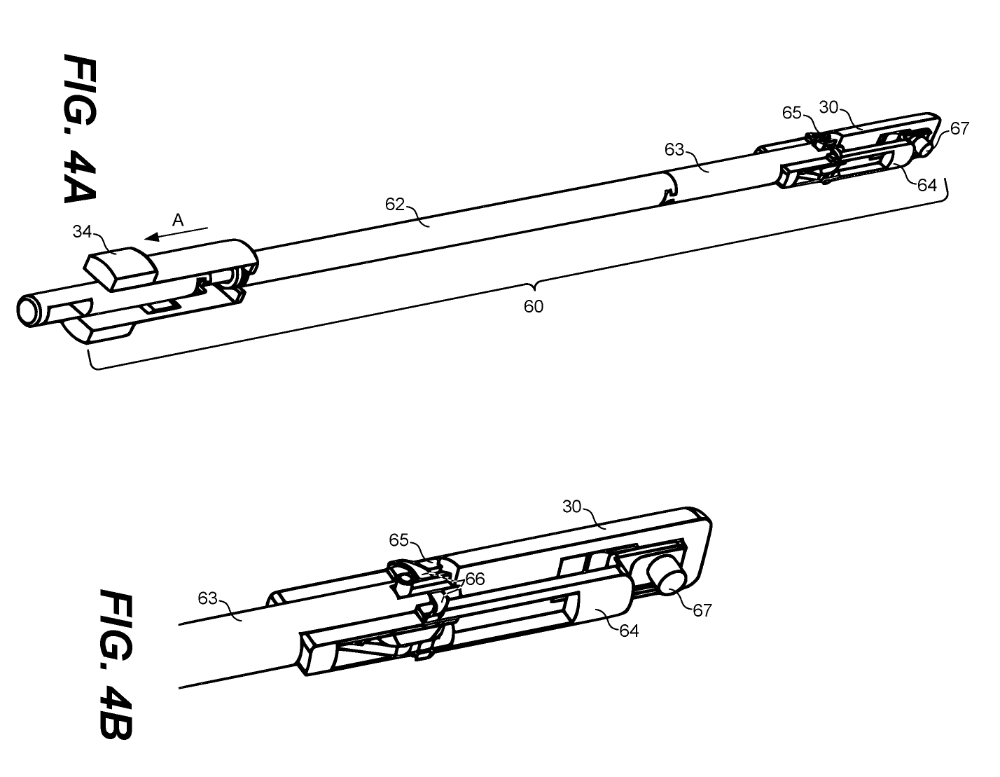

FIGS. 4A and 4B illustrate example components of a grasper door release assembly 60 of the device 10 shown in FIG. 1 according to various embodiments described herein. In FIGS. 4A and 4B, the handle assembly 12, the cannula assembly 14, and the grasper finger 31 are omitted from view to focus on other components. The grasper door release assembly 60 includes a release slide 34, a release slide tube 62, a release slide tube extension 63, and first and second sliding doors 64 and 65. In the device 10, the release slide tube 62 and the release slide tube extension 63 are enclosed within the cannula cover tube 26 shown in FIGS. 3A-3C.

The release slide 34 is clipped into a recess formed into the release slide tube 62 at one end. At its distal end, the release slide tube 62 includes a female keyhole aperture into which a mating protrusion of the release slide tube extension 63 locks into place. At its distal end, the release slide tube extension 63 includes release slide hooks 66. The release slide hooks fit into notch recesses in the first and second sliding doors 64 and 65. In FIGS. 4A and 4B, a magnet 67 is also illustrated. The magnet 67 is enclosed within the grasper finger 31, which is omitted from view in FIGS. 4A and 4B, and a similar magnet is enclosed within the grasper finger 30.

To release the magnet 67 (and the magnet enclosed within the grasper finger 30), an individual can pull or slide the release slide 61 in the direction “A” shown in FIG. 4A. In turn, the release slide 34 pulls or slides the release slide tube 62, the release slide tube extension 63, and the first and second sliding doors 64 and 65 in the same direction. When the first sliding door 64 is slid, the magnet 67 is held in place within the grasper finger 31 due to a mechanical interference, but an opening is exposed for the magnet 67 to fall out of the grasper finger 31. In other words, a void is formed in the grasper finger 31 to store the magnet 67, the first sliding door 64 covers the void, and the first sliding door 64 can be moved to permit the magnet 67 to fall out from within the grasper finger 31. A similar void is formed in the grasper finger 30 to store another magnet, the second sliding door 65 covers that void, and the second sliding door 65 can be moved to permit the other magnet to fall out from within the grasper finger 30.

As discussed above, to place the magnet 67 (and the magnet enclosed within the grasper finger 30, collectively “the magnets”) over the mitral valve 41 shown in the example heart 40 in FIG. 2, the device 10 is inserted through the apex of the heart 40 in a transapical procedure and extended up into the left ventricle 42 toward the mitral valve 41. When the distal end of the device 10 is in position with the mitral valve 41, the grasper fingers 30 and 31 are used to place the magnets over the flaps 44 and 45 of the mitral valve 41, with only the mechanical interference of the sliding doors 64 and 65 remaining between the magnets and the flaps 44 and 45. When the grasper fingers 30 and 31 and the magnets have been suitably positioned (and/or repositioned) over the flaps 44 and 45, the release slide 34 can be pulled to open the sliding doors 64 and 65, thereby removing the only remaining mechanical interference between the magnets and the flaps 44 and 45. With the sliding doors 64 and 65 moved out of the way, the magnetic attraction between the magnets pulls them together, and the magnets press and hold the flaps 44 and 45 of the mitral valve 41 together. In other embodiments of the device 10, the grasper fingers 30 and 31 can be used to place one or more staples or sutures (rather than or in addition to magnets) over the flaps 44 and 45 of the mitral valve 41.

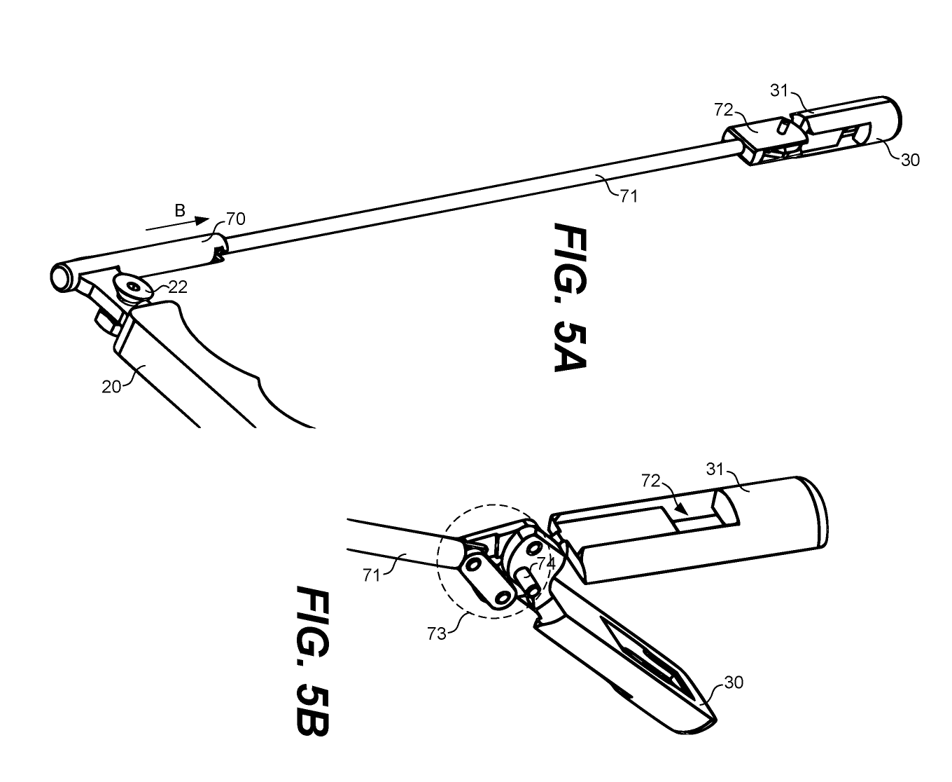

FIGS. 5A and 5B illustrate example components of a grasper assembly of the device 10 shown in FIG. 1 according to various embodiments described herein. In FIGS. 5A and 5B, the cannula assembly 14 and the grasper door release assembly 60 are omitted from view to focus on other components. Referring between FIGS. 5A and 5B, the gasper assembly includes a trigger bolt 70, a grasper rod 71, a cover 72, and pivot transfer linkage 73 partially surrounded by the cover 72.

As shown in FIGS. 5A and 5B, an end of the handle trigger 20 fits into the trigger bolt 70. The trigger bolt 70 includes a female keyhole aperture into which a mating protrusion of the grasper rod 71 locks into place. At its distal end, the grasper rod 71 includes a mating connection with the pivot transfer linkage 73, which is partially surrounded by the cover 72. The cover 72 is omitted from view in FIG. 5B to reveal the pivot transfer linkage 73. The pivot transfer linkage 73 connects to ends of the grasper fingers 30 and 31, which pivot about the grasper pivot shaft 74. In FIG. 5B, a slider pass-through 72 is visible in the grasper finger 31, because the sliding door 65 is omitted from view. The grasper finger 30 includes a similar opening to allow the sliding door 64 to pass through it.

In operation, when the handle trigger 20 is pulled, the end of the handle trigger 20 pivots about the pivot hardware 22, and pushes the trigger bolt 70 and the grasper rod 71 in the direction “B” shown in FIG. 5A. The pivot transfer linkage 73 is secured in place by the grasper pivot shaft 74 which extends through the grasper pivot shaft hole 57 in the cannula cover tube 26 as shown in FIGS. 3A-3C. Thus, when the grasper rod 71 pushes the pivot transfer linkage 73 as the handle trigger 20 is pulled, the pivot transfer linkage 73 pushes the grasper fingers 30 and 31 to pivot about the grasper pivot shaft 74 and open. In other embodiments, the handle trigger 20 can actuate the grasper fingers 30 and 31 to open and close in other ways. For example, the pivot transfer linkage 73 can include alternate pivot points, alternative arrangements of linking parts, etc. Additionally, in other embodiments, the handle trigger 20 can be used to actuate other tools, such as staple, crimper, or other tools as described herein.

While the device 10 is described as being capable of positioning and placing a pair of magnets in a transapical procedure to address MR, similar embodiments of the device 10 can be used to place one or more staples, sutures, or other bonding or connecting element to secure the flaps of the mitral valve. For example, the grasper fingers 30 and 31 could include hooks and/or loops threaded with suture material, cutting blades, etc. to secure sutures to the flaps of a mitral valve. In other embodiments, the grasper fingers 30 and 31 could include staple delivery and crimper tools, respectively, to secure sutures to the flaps of a mitral valve. However, the device 10 is not limited to those examples as it (or similar devices) can secure other connecting means to a mitral valve in a transapical procedure.

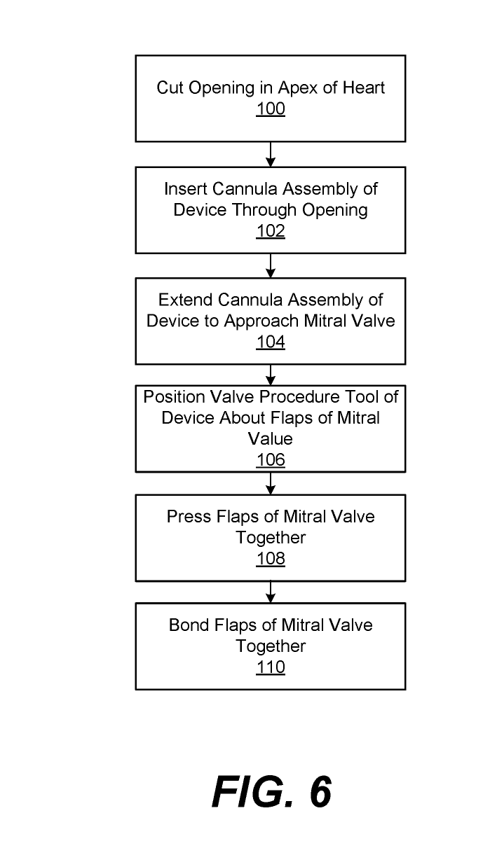

FIG. 6 illustrates an example process for a transapical procedure according to various embodiments described herein. Although the process in FIG. 6 is described below as being conducted using the device 10 shown in FIG. 1 on the heart 40 shown in FIG. 2, other devices similar to the device 10 can be used. Further, the process can be performed on other hearts, such as hearts of various animals. Additionally, although the steps in the process shown in FIG. 6 are illustrated in an order, the steps can occur in other orders depending upon the manner in which the process is conducted by the surgeon or operator.

At step 100, the process includes cutting an opening through the apex 46 of the heart 40. In one example case, the apex 46 of the heart 40 can be cut using any suitable knife, blade, or other instrument at a location to provide access to the left ventricle 42 of the heart 40. At step 102, the process includes inserting the cannula 14 of the device 10 through the opening in the apex 46 of the heart 40 and into the left ventricle 42.

At step 104, the process includes extending the cannula assembly 14 of the device 10 through the heart 40 to approach the mitral valve 41 of the heart 40. The extension and placement of the cannula 14 within the left ventricle 42 can be confirmed during the procedure using echocardiography, electrocardiography, or another suitable diagnostic technique(s) (or combinations thereof). In some cases, a camera can also be used to identify the apex 46, the mitral valve 41, and/or other features of the heart 40 during the procedure.

At step 106, the process includes positioning the valve procedure tool 16 of the device 10 about the flaps 44 and 45 of the mitral valve 41. For example, when the end of the valve procedure tool 16 is in positioned close to the flaps 44 and 45 of the mitral valve 41, the grasper fingers 30 and 31 can be opened around the flaps 44 and 45 by pulling on the handle trigger 20. At the same time, the relative positions of the device 10, the grasper fingers 30 and 31, and the flaps 44 and 45 can be confirmed using echocardiography, electrocardiography, or any other suitable diagnostic technique(s). Additionally, the cannula 14 assembly, including the grasper fingers 30 and 31, can be rotated into position around the flaps 44 and 45 using the cannula rotator knob 24.

After the grasper fingers 30 and 31 have been positioned around the flaps 44 and 45, the process includes pressing at least a portion of the flaps 44 and 45 of the mitral valve 41 together. For example, the handle trigger 20 can be released to close the grasper fingers 30 and 31 over the flaps 44 and 45 (e.g., over central portions of the flaps 44 and 45). In some cases, with the grasper fingers 30 and 31 closed over the flaps 44 and 45, the performance of the mitral valve 41 can be evaluated using echocardiography. If needed, the grasper fingers 30 and 31 can be repositioned over the flaps 44 and 45 again to achieve better results. For example, the grasper fingers 30 and 31 may be repositioned to hold the flaps 44 and 45 together near the center of the flaps 44 and 45.

When the grasper fingers 30 and 31 are suitably positioned and the flaps 44 and 45 of the mitral valve 41 are pressed together, the process includes bonding at least a portion of the flaps 44 and 45 of the mitral valve 41 together. The bonding can include applying a bonding element to the flaps 44 and 45 of the mitral valve 41 using the valve procedure tool. For example, the magnets in the grasper fingers 30 and 31 can be released using the release slide 34. As described above, when the release slide 34 is pulled back, doors in the grasper fingers 30 and 31 are also pulled or slid back, releasing the magnets out from within the grasper fingers 30 and 31. The magnets are magnetically attracted to each other and hold (e.g., press or bond) the flaps 44 and 45 together in a manner similar to that achieved by the Alfieri stitch. In other embodiments of the device 10, the grasper fingers 30 and 31 can be used to bond the flaps 44 and 45 of the mitral valve 41 together by placing one or more staples or sutures (rather than or in addition to magnets) over the flaps of the mitral valve.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

At least the following is claimed:

1. A device for a transapical procedure to mitigate mitral regurgitation, comprising:
a handle assembly;
a cannula assembly; and
a valve procedure tool disposed at a distal end of the cannula assembly to position and place a bonding element to flaps of a mitral valve during the transapical procedure.

2. The device according to claim 1, wherein the handle assembly comprises a handle grip, a handle trigger, and pivot hardware that secures the handle trigger in position as a lever with the handle grip.

3. The device according to claim 1, wherein the cannula assembly comprises a cannula cover tube and a cannula rotator knob to rotate the cannula cover tube and the valve procedure tool.

4. The device according to claim 1, wherein the valve procedure tool comprises at least one grasper finger to place the bonding element to the flaps of the mitral valve during the transapical procedure.

5. The device according to claim 1, wherein:
the bonding element comprises at least one of a magnet, a staple, or suture filaments; and
the valve procedure tool comprises a plurality of grasper fingers to place the bonding element over the flaps of the mitral valve during the transapical procedure.

6. The device according to claim 1, wherein:
the bonding element comprises a plurality of magnets; and
the valve procedure tool comprises a plurality of grasper fingers to place the plurality of magnets over the flaps of the mitral valve during the transapical procedure.

7. The device according to claim 6, wherein:
the plurality of magnets are enclosed within the plurality of grasper fingers; and
the device further comprises a sliding door in each of the plurality of grasper fingers to release the plurality of magnets from the device over the flaps of the mitral valve during the transapical procedure.

8. A device for a procedure, comprising:
a handle assembly comprising a handle grip, a handle trigger, and pivot hardware that secures the handle trigger in position as a lever with the handle grip;
a cannula assembly; and
a valve procedure tool disposed at a distal end of the cannula assembly to position and place a bonding element during the procedure.

9. The device according to claim 8, wherein the cannula assembly comprises a cannula cover tube and a cannula rotator knob to rotate the cannula cover tube and the valve procedure tool.

10. The device according to claim 8, wherein the valve procedure tool comprises at least one grasper finger to place the bonding element during the procedure.

11. The device according to claim 8, wherein:
the bonding element comprises at least one of a magnet, a staple, or suture filaments; and
the valve procedure tool comprises a plurality of grasper fingers to place the bonding element during the procedure.

12. The device according to claim 8, wherein:
the bonding element comprises a plurality of magnets; and
the valve procedure tool comprises a plurality of grasper fingers to place the plurality of magnets during the procedure.

13. The device according to claim 12, wherein:
the plurality of magnets are enclosed within the plurality of grasper fingers; and
the device further comprises a sliding door in each of the plurality of grasper fingers to release the plurality of magnets from the device during the procedure.

14. The device according to claim 8, wherein the procedure comprises a transapical procedure to mitigate mitral regurgitation.

15. A transapical procedure to mitigate mitral regurgitation, comprising:
inserting a cannula assembly of a device into an apex opening in a heart;
extending the cannula assembly of the device through the heart to approach a mitral valve of the heart; and
positioning a valve procedure tool about flaps of a mitral valve.

16. The transapical procedure according to claim 15, wherein positioning the valve procedure tool comprises rotating the valve procedure tool about the flaps of the mitral valve.

17. The transapical procedure according to claim 15, wherein, after positioning the valve procedure tool, the procedure further comprises pressing at least a portion of the flaps of the mitral valve together.

18. The transapical procedure according to claim 17, wherein, pressing at least the portion of the flaps of the mitral valve together, the procedure further comprises bonding at least a portion of the flaps of the mitral valve together by applying a bonding element to the flaps of the mitral valve using the valve procedure tool.

19. The transapical procedure according to claim 18, wherein:
the bonding element comprises at least one of a magnet, a staple, or suture filaments; and
the valve procedure tool comprises a plurality of grasper fingers to place the bonding element over the flaps of the mitral valve during the transapical procedure.

20. The transapical procedure according to claim 19, wherein:
the bonding element comprises a plurality of magnets; and
the valve procedure tool comprises a plurality of grasper fingers to place the plurality of magnets over the flaps of the mitral valve during the transapical procedure.
